(19) United States
(12) Patent Application Publication
(10) Pub. No.: US 2026/0191546 A1
(43) Pub. Date: Jul. 9, 2026

(54) DUAL-LUMEN INTRAVASCULAR ULTRASOUND CATHETER

(71) Applicant: University of Pittsburgh – Of the Commonwealth System of Higher Education, Pittsburgh, PA (US)

(72) Inventor: Catalin Toma, Pittsburgh, PA (US)

(21) Appl. No.: 19/128,475

(22) PCT Filed: Nov. 9, 2023
(86) PCT No.: PCT/US2023/079235

§ 371 Date: May 8, 2025

Related U.S. Application Data
(60) Provisional application No. 63/423,989, filed on Nov. 9, 2022.

(51) Int. Cl. A61B 17/22; A61B 8/00; A61B 8/08; A61B 8/12; A61B 17/00; A61B 90/00; A61M 25/00; A61M 25/01

(52) U.S. Cl. CPC A61B 17/22; A61B 8/0891; A61B 8/12; A61B 8/445; A61B 8/4461; A61B 8/5223; A61B 17/00234; A61B 90/36; A61M 25/0029; A61M 25/003; A61M 25/01; A61B 2017/00305; A61B 2017/00778; A61B 2017/00924; A61B 2017/22038; A61B 2017/22095; A61B 2090/3784; A61M 2025/0037; A61M 2025/0042; A61M 2205/3375; A61M 2210/12

(57) ABSTRACT

A system hereof includes a catheter including a first lumen passing through the length of the catheter to a distal end thereof and having an exit port on the distal end of the catheter and a second lumen adjacent to the first lumen. The second lumen includes a port spaced proximally from the exit port of the first lumen. An ultrasound transparent section of the catheter in the vicinity of the port of the second lumen. The ultrasound transparent section being transparent to ultrasound energy. The system further includes an intravascular ultrasound imaging element including an ultrasonic transducer on a distal end thereof. The first lumen has a diameter sufficiently large to allow passage of the rotational, intravascular ultrasound imaging element therethrough to position the ultrasonic transducer adjacent to the ultrasound transparent section.

### CROSS-REFERENCE TO RELATED APPLICATIONS

This application claims benefit of U.S. Provisional Patent Application Ser. No. 63/423,989, filed Nov. 9, 2022, the disclosure of which is incorporated herein by reference.

### BACKGROUND

The following information is provided to assist the reader in understanding technologies disclosed below and the environment in which such technologies may typically be used. The terms used herein are not intended to be limited to any particular narrow interpretation unless clearly stated otherwise in this document. References set forth herein may facilitate understanding of the technologies or the background thereof. The disclosure of all references cited herein are incorporated by reference.

In general terms, a coronary intervention involves crossing a completely blocked artery with a guidewire over which angioplasty and stenting are performed. Such an intervention is typically accomplished with guidance based upon angiographic imaging. There are certain situations, however, in which wire guidance cannot be provided based on the angiographic image. For example, a target area may either completely occluded with no dye penetration, or the exact angulation/origin of the branch to be accessed may be unclear. Various aspects of such procedures are discussed in, for example, U.S. Pat. Nos. 7,179,270, 8,202,246, and 9,814,862, as well as Wu, E. B., et al, Advances in CrossBoss/Stingray use in antegrade dissection reentry from the Asia Pacific Chronic Total Occlusion Club, Catheter Cardiovasc Interv., 96:1423-1433 (2020), the disclosures of which are incorporated herein by reference. The current technology in antegrade dissection and re-entry is, for example, represented by the use of a CROSSBOSS™ catheter to advance in the subintimal space beyond an occlusion and a STINGRAY re-entry system (both available from Boston Scientific Corporation of Marlborough, Massachusetts US), which have increased the success of re-entry. However, a number of problems persist.

Intravascular ultrasound (IVUS) imaging has been used in connection with a catheter including a dedicated reentry needle and a phased array IVUS in the PIONEER PLUS IVUS-guided reentry catheter available from Philips of Andover, Massachusetts US. In that catheter, a phased array IVUS is used and a guide wire can be passed through the center of the array which is peripherally positioned in the catheter lumen. The catheter is very large in size and is not usable in connection with, for example, relatively small vessels such as coronary vessels.

Although a number of advances have been made in such vascular interventional procedures, it remains desirable to develop improved devices, systems, and method for coronary and other intervention.

### SUMMARY

In one aspect, a system hereof includes a catheter including a first lumen passing through the length of the catheter to a distal end thereof and having an exit port on the distal end of the catheter and a second lumen adjacent to the first lumen. The second lumen includes (and typically terminates with) a (side) port spaced proximally from the exit port of the first lumen. An ultrasound transparent section of the catheter in the vicinity of the port of the second lumen is transparent to ultrasound energy. The system further includes an intravascular ultrasound imaging element including an ultrasound transducer on a distal end thereof. The first lumen has a diameter sufficiently large to allow passage of the intravascular ultrasound imaging element therethrough to position the ultrasonic transducer adjacent to the ultrasound transparent section.

In a number of embodiments, the catheter is dimensioned to pass or travel through a subintimal space of a blood vessel (axially or along the length of the subintimal space). In a number of embodiments, the diameter of the first lumen decreases in the vicinity of the exit port thereof to prevent passage of the intravascular ultrasound imaging element therethrough. The diameter of the second lumen is, in a number of embodiments, suitable to pass a guidewire therethrough for reentry into a true lumen of the blood vessel.

The intravascular ultrasound imaging element is a rotational intravascular ultrasound imaging element in a number of embodiments. In a number of embodiments, the catheter has an oval cross-sectional shape.

In another aspect, a catheter includes a first lumen passing through the length of the catheter to a distal end thereof and having an exit port on the distal end of the catheter and a second lumen adjacent to the first lumen. The second lumen includes (and typically terminates with) a (side) port spaced proximally from the exit port of the first lumen, and an ultrasound transparent section of the catheter in the vicinity of the port of the second lumen is transparent to ultrasound energy. The first lumen has a diameter sufficiently large to allow passage of an intravascular ultrasound imaging element therethrough to position an ultrasonic transducer of the intravascular ultrasound imaging element adjacent to the ultrasound transparent section. In general, the ultrasound transparency of the ultrasound transparent section is maximized (for example, at least 95% or at least 99%).

In a number of embodiments, the catheter is dimensioned to pass through a subintimal space of a blood vessel. In a number of embodiments, the diameter of the first lumen decreases in the vicinity of the exit port thereof to prevent passage of the intravascular ultrasound imaging element therethrough. The diameter of the second lumen is, in a number of embodiments, suitable to pass a guidewire therethrough for reentry into a true lumen of the blood vessel.

As described above, the intravascular ultrasound imaging element is a rotational intravascular ultrasound imaging element in a number of embodiments. In a number of embodiments, the catheter has an oval cross-sectional shape.

In a further aspect, a method includes extending a catheter into a blood vessel over a guidewire. The catheter includes a first lumen passing through the length of the catheter to a distal end thereof and having an exit port on the distal end of the catheter. The guidewire passes through the first lumen during extension of the catheter into the blood vessel. The catheter further includes a second lumen adjacent the first lumen. The second lumen includes (and typically terminates with) a (side) port spaced proximally from the exit port of the first lumen. An ultrasound transparent section of the catheter in the vicinity of the port of the second lumen is transparent to ultrasound energy. The method further includes removing the guidewire and extending an intravascular ultrasound imaging element, which includes an ultrasound transducer on a distal end thereof, through the first lumen to position the ultrasonic transducer in adjacent to the ultrasound transparent section. The method further includes extending a second wire through the second lumen and using the ultrasound image via the intravascular ultrasound imaging element to direct the re-entry wire as it is extended through the port of the second lumen to the true lumen of the vessel distal to the lesion.

In a number of embodiments, the catheter is extended to pass through a subintimal space of a blood vessel. In a number of embodiments, the diameter of the first lumen decreases in the vicinity of the exit port thereof to prevent passage of the intravascular ultrasound imaging element therethrough. The second wire may, for example, be extended through the port of the second lumen to reenter into a true lumen of the blood vessel. In a number of embodiments, the catheter enters the subintimal space on a proximal side of an occlusion in the blood vessel and the second wire reenters the true lumen on a distal side of the occlusion.

Once again, as described above, the intravascular ultrasound imaging element is a rotational intravascular ultrasound imaging element in a number of embodiments. In a number of embodiments, the catheter has an oval cross-sectional shape.

The method may further include performing at least one of retraction, advancement, or rotation of the catheter to determine a position at which to direct the second wire as it is extended through the port of the second lumen. In a number of embodiments, the position is a reentry position from the subintimal space to the true lumen and the position is based on at least one of a determination of size of the true lumen and a degree of calcification on the wall of the true lumen as determined by the ultrasound image. The method hereof may be used in or form a part of a process of (i) true lumen reentry, (ii) side branch access, (iii) puncture of an ambiguous or difficult to cross proximal cap, (iv) reverse controlled antegrade and retrograde tracking, (v) identifying a subintimal hematoma, (vi) management of the subintimal hematoma, (vii) identifying an intramural hematoma, or (viii) management of the intramural hematoma.

In still a further aspect, a method of use of a catheter is provided wherein the catheter include a first lumen passing through the length of the catheter to a distal end thereof and having an exit port on the distal end of the catheter, and a second lumen adjacent to the first lumen, the second lumen comprising a port spaced proximally from the exit port of the first lumen, wherein an ultrasound transparent section of the catheter in the vicinity of the port of the second lumen is transparent to ultrasound energy, wherein the first lumen has a diameter sufficiently large to allow passage of an intravascular ultrasound imaging element therethrough to position an ultrasonic transducer of the intravascular ultrasound imaging element adjacent to the ultrasound transparent section. The method includes extending the catheter over a guidewire, removing the guidewire; extending the intravascular ultrasound imaging element including the ultrasonic transducer on a distal end thereof through the first lumen to position the ultrasonic transducer adjacent the ultrasound transparent section, extending a second wire through the second lumen; and creating an ultrasound image via the intravascular ultrasound imaging element to direct the second wire as it is extended through the port of the second lumen.

The present devices, systems, and methods along with the attributes and attendant advantages thereof, will best be appreciated and understood in view of the following detailed description taken in conjunction with the accompanying drawings.

### BRIEF DESCRIPTION OF THE DRAWINGS

FIG. 1A illustrates schematically a side view of a proximal and a distal end section of an embodiment of a dual-lumen catheter hereof.

FIG. 1B illustrates schematically a cross-sectional view of the catheter of FIG. 1A.

FIG. 1C illustrates schematically the passage of a distal end section of the catheter of FIG. 1A into the subintimal space to a point beyond a coronary total occlusion or CTO to that a wire can reenter the true artery lumen via a port in the catheter beyond the CTO.

FIG. 1D illustrates ultrasound-imaging-guided puncture of an ambiguous/difficult to cross proximal cap using a dual-lumen catheter hereof positioned in a side branch at the proximal cap.

FIG. 1E illustrates ultrasound-imaging-guided identification and management of an intramural hematoma using a dual-lumen catheter hereof.

FIG. 1F illustrates distal branch access using a dual-lumen catheter hereof wherein the catheter is positioned in a side branch at the proximal cap and a wire exiting the port of the second lumen is positioned at the bifurcation towards the other branch.

FIG. 1G illustrates ultrasound-imaging-guided positioning of a wire exiting the port of the second lumen of a dual-lumen catheter hereof relative to a retrograde knuckle/microcatheter in a subintimal space created by the retrograde knuckle/microcatheter in a reverse controlled antegrade and retrograde tracking (CART) procedure.

FIG. 2A illustrates a perspective view of another embodiment of the distal end section of a dual-lumen catheter hereof.

FIG. 2B illustrates an enlarged perspective view of a portion of the distal end section of the catheter of FIG. 2A.

FIG. 2C illustrates another enlarged perspective view of a portion of the distal end section of the catheter of FIG. 2A.

FIG. 2D illustrates an enlarged cutaway view of the catheter of FIG. 2A in the vicinity of the opening, passage or exit of the second, smaller lumen.

### DETAILED DESCRIPTION

It will be readily understood that the components of the embodiments, as generally described and illustrated in the figures herein, may be arranged and designed in a wide variety of different configurations in addition to the described representative embodiments. Thus, the following more detailed description of the representative embodiments, as illustrated in the figures, is not intended to limit the scope of the embodiments, as claimed, but is merely illustrative of representative embodiments.

Reference throughout this specification to “one embodiment” or “an embodiment” (or the like) means that a particular feature, structure, or characteristic described in connection with the embodiment is included in at least one embodiment. Thus, the appearance of the phrases “in one embodiment” or “in an embodiment” or the like in various places throughout this specification are not necessarily all referring to the same embodiment.

Furthermore, described features, structures, or characteristics may be combined in any suitable manner in one or more embodiments. In the following description, numerous specific details are provided to give a thorough understanding of embodiments. One skilled in the relevant art will recognize, however, that the various embodiments can be practiced without one or more of the specific details, or with other methods, components, materials, et cetera. In other instances, well known structures, materials, or operations are not shown or described in detail to avoid obfuscation.

As used herein and in the appended claims, the singular forms “a,” “an”, and “the” include plural references unless the context clearly dictates otherwise. Thus, for example, reference to “an exit port” includes a plurality of such exit ports and equivalents thereof known to those skilled in the art, and so forth, and reference to “the exit port” is a reference to one or more such exit ports and equivalents thereof known to those skilled in the art, and so forth. Recitation of ranges of values herein are merely intended to serve as a shorthand method of referring individually to each separate value falling within the range. Unless otherwise indicated herein, each separate value, as well as intermediate ranges, are incorporated into the specification as if individually recited herein. All methods described herein can be performed in any suitable order unless otherwise indicated herein or otherwise clearly contraindicated by the text.

In a number of embodiments, a catheter hereof is a dual lumen intravascular ultrasound (IVUS) catheter. Catheters hereof may, for example, be functional to improve or optimize the process of true lumen reentry during percutaneous coronary intervention (PCI) for chronic total occlusion (CTO), sometimes referred to as CTO PCI, when an antegrade dissection reentry approach is used. As described above, the current technology in this space is represented by the STINGRAY catheter which represents an important component that has increased the success of re-entry. However, a number of problems persist. For example, there is difficulty in visualizing and choosing the best spot for successful reentry. Using an imaging system to guide reentry has multiple advantages over the current technology. Although a number of embodiments of devices, systems, and methods hereof are discussed in connection with coronary disease, the devices systems and methods hereof may also, for example, be used in connection with peripheral vascular disease.

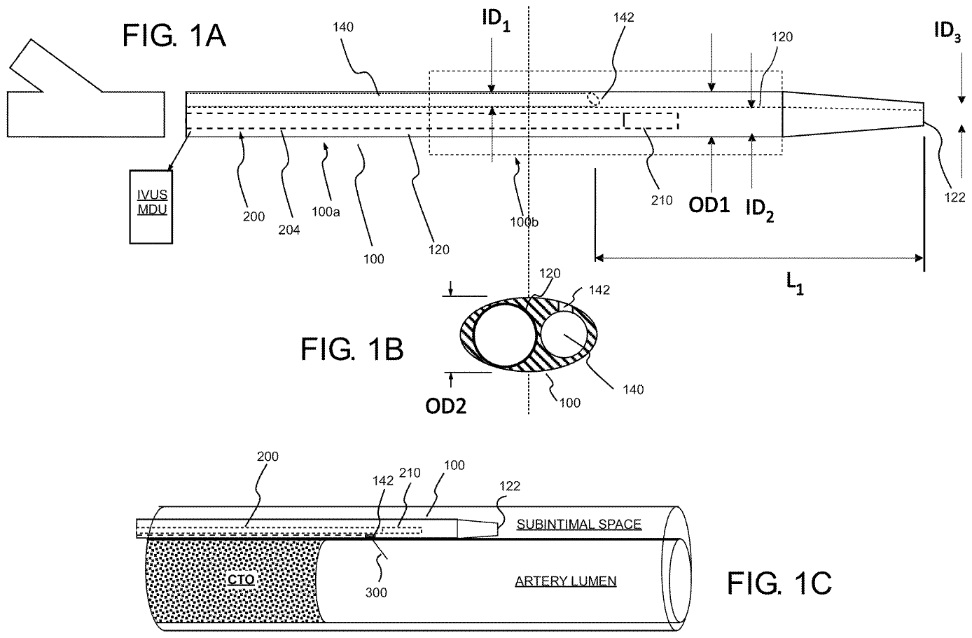

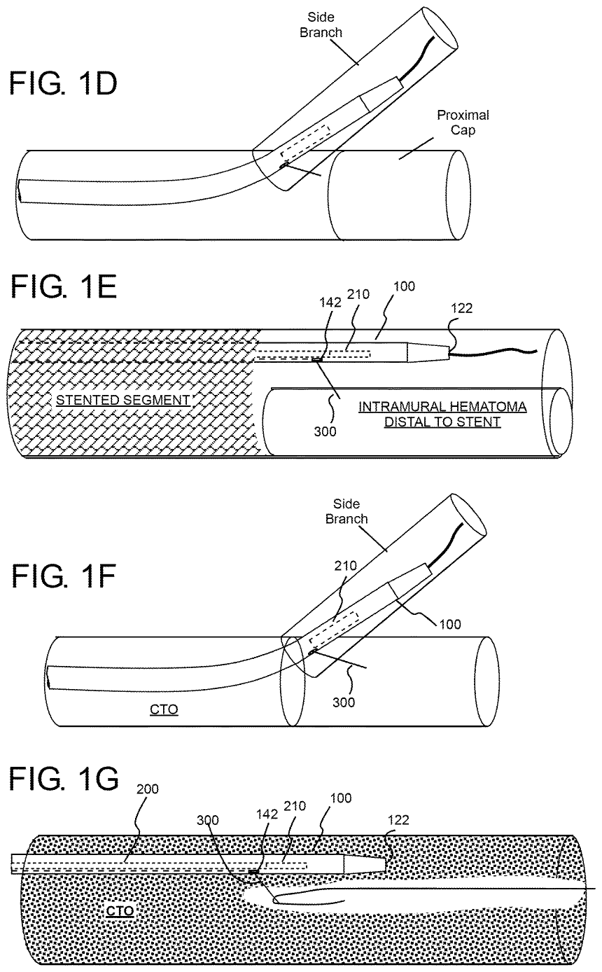

In a number of embodiments hereof, as, for example, illustrated schematically in FIGS. 1A and 1B, a dual-lumen, an intravascular ultrasound (IVUS) catheter or microcatheter 100 hereof includes a first section 100a, which may be a braided section. Microcatheter 100 further includes a second section 100b (illustrated within a broken-lined rectangle in FIG. 1A), which is an ultrasound transparent section (formed, for example, from a polymer such as a polyethylene). A central lumen 120 spans the entire length of catheter 100. Central lumen 120 includes or is in connection with, a port or opening 122 (an over-the-wire or OTW port) at a distal end of microcatheter 100. Lumen 120 may, for example, be tapered towards opening 122 (that is, towards its distal end or tip). Lumen 120 provides for both the advancement of microcatheter 100 in a distal reentry zone (see, for example, FIG. 1C) over a wire (not shown) and also, following the wire removal, it can accommodate an ultrasound element 200 (illustrated schematically in FIG. 1A). In a number of embodiments, ultrasound element 200 is a single-element, rotating or spinning IVUS probe. A representative example of suitable ultrasound technology for use herein is that used in the coronary IVUS OPTICROSS™ catheter technology, available from Boston Scientific Corporation of Marlborough, MA, USA. In general, a single-element, rotating or spinning IVUS probe can provide a better image than a phased array of ultrasound elements used in some intravascular ultrasound technologies. It is not possible to position a guide wire and a rotating IVUS probe in the same lumen at the same time. Ultrasound element 200 may, for example, include a hi-torque, flexible, rotating drive cable 204 with a radial looking 40 MHz ultrasonic transducer 210 at the distal tip thereof. Although rotational IVUS probes are used in a number of embodiments hereof, other intravascular ultrasound elements/systems may be used.

In the illustrated embodiment, a second lumen 140 also spans substantially the entire length of microcatheter 100. However, second lumen 140 includes an exit port 142, typically at the terminus of second lumen 140, proximal to the tip of microcatheter 100 on the side thereof in the same area of microcatheter 100 where transducer 210 of the IVUS catheter 200 resides. While port 122 is oriented in a generally axial direction, exit port or side exit port 142 is oriented generally radially or with a radial component and upward (in, for example, the orientation of FIG. 1B) through the outer wall of microcatheter 100. IVUS element/transducer 210 can be advanced beyond exit port 142 (which may be marked with ultrasound opaque material), for example, up to 5 mm more distal toward the exit port 142. IVUS element/transducer 210 can be moved manually or under software control to provide a desired imaging profile over a defined or determined length of the catheter or IVUS element/transducer 210 can be locked in position.

In a number of embodiments, catheter 100 is sufficiently long (for example, approximately 120 cm in length) to extend, for example, from the wrist or from the groin to the heart. Catheter 100 is otherwise dimensioned to, for example, pass through a subintimal space or portion of a coronary artery as illustrated in FIG. 1C. A number of representative dimensions for the representative embodiment of microcatheter 100 are set forth in FIGS. 1A and 1B wherein ID1 (which is the inner diameter of second lumen 140) is approximately 0.016 in. (0.041 cm); ID2 (which is the inner diameter of first lumen 120) is approximately 0.025 in. (0.064 cm); ID3 (which is the inner diameter of port/opening 122 is approximately 0.016 in. (0.041 cm); OD1 (which is the outer diameter of the major diameter/width of the oval catheter) is 0.059 in. (0.15 cm); OD2 (which is the outer diameter of the minor diameter/heigh of the oval catheter) is 0.0394 in. (0.1 cm); and L1 (which is the length from port 142 to port '122) is 0.78 in. (1.98 cm). First lumen 120, the larger of the two lumens, must have a diameter suitable to pass ultrasound element/fiber 200 therethrough. Typically, the diameter of a lumen is closely matched to the device(s) to be passed therethrough. An oval cross-section shape of microcatheter 100 (see FIG. 1B) facilitates proper orientation of microcatheter 100 when passing through a generally half-moon shaped slit or opening in the subintimal space. In a number of embodiments, the maximum major diameter or width of catheter 100 is approximately 0.175 cm, 0.15 cm or 0.12 cm. In a number of embodiments, the maximum minor diameter or height of catheter 100 is approximately 0.1 cm or approximately 0.75 cm. In a number of embodiments, the maximum diameter of first lumen 120 is approximately 0.03 in. (0.0762 cm) or 0.025 in (0.064 cm). In a number of embodiments, the maximum diameter of second lumen 140 is approximately 0.018 in. (0.0457 cm). As used herein, the terms “approximately”, “generally”, and the like refer to a value within a range of +/−10 percent (and more typically within a range of +/−5% of the state value (or values if a range is set forth).

In an embodiment of a mode of use of microcatheter 100, microcatheter 100 is advanced (via first lumen 120 over a guidewire (not shown; which extends, for example, through a subintimal portion of an artery) to a distal reentry zone. Subintimal hematomas may be identified and managed (via aspiration through port 122). Subsequently, the guidewire is removed and the core element 200 of an IVUS catheter is advanced through first lumen 120 to the tip and connected to the IVUS motordrive unit (MDU). In a number of embodiments, lumen 120 tapers toward the tip to, for example, approximately 0.014 to 0.016 inches (0.036 to 0.041 cm), thereby preventing inadvertent exit of IVUS catheter 200 through the distal port a distal end 122 of microcatheter 100. Subsequently, a stiff, pre-shaped coronary wire 300 (see FIG. 1C; such as HORNET™ 14 guidewire (nominal diameter of 0.014 in or 0.0356 cm), available from Boston Scientific Corporation) may be advanced through second lumen 140, which can be directed based on the IVUS data/imaging, to find a suitable or optimal spot for a reentry zone (for example, aligned with a suitable true lumen size and having an acceptable amount of calcification) for reentry into the true artery lumen. Second lumen 140 and exit port 142 are apparent on the resultant image. Markers may be added at one or more positions to facilitate localization using ultrasound and x-ray imaging/localization. The puncture may be done under live IVUS imaging as the wire will be clearly visible on the ultrasound image. If the wire exit position does not correspond to an acceptable or optimal re-entry position or spot, microcatheter 100 can be rotated and/or advanced back and forth to achieve acceptable or optimal positioning (for example, based on a determination of size of true lumen and a degree of calcification on the wall of the true lumen as determined by the ultrasound image). In embodiments in which catheter 100 is generally oval in shape, the self-orientation of the catheter will typically mean that port 142 is oriented radially outward or radially inward. Once such procedures have been completed, the same IVUS rotating element can be reinserted into a regular IVUS sheath allowing for traditional IVUS imaging of the final result and procedure optimization.

FIGS. 1D through 1G illustrate a number of alternative uses of catheter 100 hereof. FIG. 1D, for example, illustrates ultrasound-imaging-guided puncture of an ambiguous or difficult to cross proximal cap using a catheter 100, which is positioned in a side branch at the proximal cap. Wire 300 is illustrated exiting port 142 toward the proximal cap. FIG. 1E illustrates ultrasound-imaging-guided identification and management of an intramural hematoma using a dual-lumen catheter hereof. The intramural hematoma is illustrated distal to a stented segment. The hematoma is punctured and evacuated under ultrasound guidance. FIG. 1F illustrates distal branch access wherein catheter 100 is positioned in a side branch at the proximal cap and wire 300 exits port 142 of second lumen 140 to be positioned at the bifurcation towards the other branch. FIG. 1G illustrates positioning of wire 300, exiting port 142 of second lumen 140 of catheter 100, relative to a retrograde knuckle/microcatheter in a subintimal space created by the retrograde knuckle/microcatheter in a reverse controlled antegrade and retrograde tracking (CART) procedure. In all procedures using catheter 100, angiographic imaging is improved without increasing radiation. Further the use of imaging contrast may be reduced.

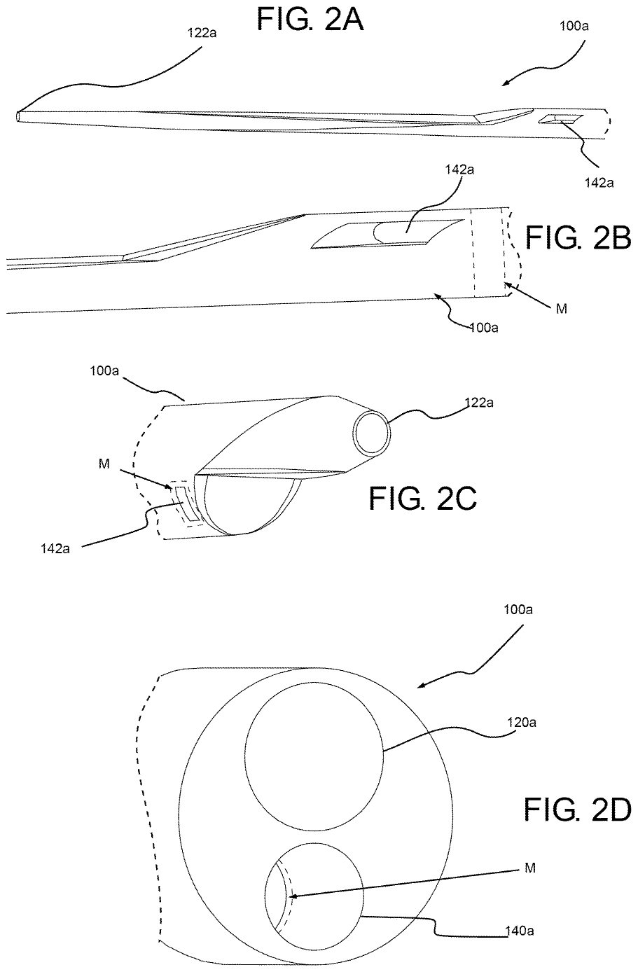

FIGS. 2A through 2D illustrate another embodiment of catheter or microcatheter 100a hereof which function in a similar or the same manner as catheter 100. Elements or components of catheter 100a are referenced similarly to like elements or components of catheter 100 with addition of the designation “a” to the end of the reference character. Markers to facilitate ultrasound localization are designated with the reference character M.

In addition to facilitating reentry in a coronary chronic total occlusion (CTO) or a substantially chronic total occlusion, the devices, system, and methods hereof have a number of other potential uses. For example, the devices, system, and methods hereof may, for example, be used for IVUS guided proximal cap puncture in the context of CTO percutaneous coronary intervention (PCI). The devices, system, and methods hereof may, for example, also be used for side branch access as well as for distal coronary wall hematoma puncture and evacuation.

The foregoing description and accompanying drawings set forth a number of representative embodiments at the present time. Various modifications, additions and alternative designs will, of course, become apparent to those skilled in the art in light of the foregoing teachings without departing from the scope hereof, which is indicated by the following claims rather than by the foregoing description. All changes and variations that fall within the meaning and range of equivalency of the claims are to be embraced within their scope.

1. A system, comprising:
a catheter comprising
a first lumen passing through the length of the catheter to a distal end thereof and having an exit port on the distal end of the catheter, and
a second lumen adjacent to the first lumen, the second lumen comprising a port spaced proximally from the exit port of the first lumen, wherein an ultrasound transparent section of the catheter in the vicinity of the port of the second lumen is transparent to ultrasound energy; and
an intravascular ultrasound imaging element comprising an ultrasonic transducer on a distal end thereof,
wherein the first lumen has a diameter sufficiently large to allow passage of the intravascular ultrasound imaging element therethrough to position the ultrasonic transducer adjacent to the ultrasound transparent section.

2. The system of claim 1 wherein the catheter is dimensioned to pass through a subintimal space of a blood vessel.

3. The system of claim 1 wherein the diameter of the first lumen decreases in the vicinity of the exit port thereof to prevent passage of the intravascular ultrasound imaging element therethrough.

4. The system of claim 2 wherein the diameter of the second lumen is suitable to pass a guidewire therethrough for reentry into a true lumen of the blood vessel.

5. The system of claim 1 wherein the intravascular ultrasound imaging element is a rotational intravascular ultrasound imaging element.

6. The system of claim 1 wherein the catheter has an oval cross-sectional shape.

7. The system of claim 1 wherein the port of the second lumen is located at the terminus of the second lumen and exits a side of the catheter.

8. A catheter, comprising:
a first lumen passing through the length of the catheter to a distal end thereof and having an exit port on the distal end of the catheter, and
a second lumen adjacent to the first lumen, the second lumen comprising a port spaced proximally from the exit port of the first lumen, wherein an ultrasound transparent section of the catheter in the vicinity of the port of the second lumen is transparent to ultrasound energy;
wherein the first lumen has a diameter sufficiently large to allow passage of an intravascular ultrasound imaging element therethrough to position an ultrasonic transducer of the intravascular ultrasound imaging element adjacent to the ultrasound transparent section.

9. The catheter of claim 8 wherein the catheter is dimensioned to pass through a subintimal space of a blood vessel.

10. The catheter of claim 8 wherein the diameter of the first lumen decreases in the vicinity of the exit port thereof to prevent passage of the intravascular ultrasound imaging element therethrough.

11. The catheter of claim 9 wherein the diameter of the second lumen is suitable to pass a wire therethrough for reentry into a true lumen of the blood vessel.

12. The catheter of claim 8 wherein the intravascular ultrasound imaging element is a rotational intravascular ultrasound imaging element.

13. The catheter of claim 8 wherein the catheter has an oval cross sectional shape.

14. The catheter of claim 8 wherein the port of the second lumen is located at the terminus of the second lumen and exits a side of the catheter.

15. A method, comprising:
extending a catheter into a blood vessel over a guidewire, the catheter comprising
a first lumen passing through the length of the catheter to a distal end thereof and having an exit port on the distal end of the catheter, the guidewire passing through the first lumen during extension of the catheter into the blood vessel, and
a second lumen adjacent to the first lumen, the second lumen comprising a port spaced proximally from the exit port of the first lumen, wherein an ultrasound transparent section of the catheter in the vicinity of the port of the second lumen is transparent to ultrasound energy;
removing the guidewire;
extending an intravascular ultrasound imaging element comprising an ultrasonic transducer on a distal end thereof through the first lumen to position the ultrasonic transducer adjacent the ultrasound transparent section;
extending a second wire through the second lumen; and
creating an ultrasound image via the intravascular ultrasound imaging element to direct the second wire as it is extended through the port of the second lumen.

16. The method of claim 15 wherein the catheter is extended to pass through a subintimal space of the blood vessel.

17. The method of claim 15 wherein the diameter of the first lumen decreases in the vicinity of the exit port thereof to prevent passage of the intravascular ultrasound imaging element therethrough.

18. The method of claim 16 wherein the second wire is extended through the port of the second lumen to reenter into a true lumen of the blood vessel.

19. The method of claim 18 wherein the catheter enters the subintimal space on a proximal side of an occlusion in the blood vessel and the second wire reenters the true lumen on a distal side of the occlusion.

20. -22. (canceled)

23. The method of claim 15 further comprising performing at least one of retraction, advancement, or rotation of the catheter to determine a position at which to direct the second wire as it is extended through the port of the second lumen.

24. The method of claim 23 wherein the position is a reentry position from the subintimal space to the true lumen and the position is based on at least one of a determination of size of the true lumen and a degree of calcification on the wall of the true lumen as determined by the ultrasound image.

25. (canceled)

26. The method of claim 15 used in a process of true lumen reentry, side branch access, puncture of an ambiguous or difficult to cross proximal cap, reverse controlled antegrade and retrograde tracking, identifying a subintimal hematoma, management of the subintimal hematoma, identifying an intramural hematoma, of management of the intramural hematoma.

27. (canceled)
